(19) United States
(12) Patent Application Publication
(10) Pub. No.: US 2021/0322310 A1
(43) Pub. Date: Oct. 21, 2021

(54) Inhalable Formulation of a Solution Containing Tiotropium Bromide

(71) Applicants: Cai Gu Huang, Shrewsbury, MA (US); Xiaoqian Wang, Shanghai (CN)

(72) Inventors: Cai Gu Huang, Shrewsbury, MA (US); Xiaoqian Wang, Shanghai (CN)

(73) Assignee: Cai Gu Huang, Shrewsbury, MA (US)

(21) Appl. No.: 17/231,777

(22) Filed: Apr. 15, 2021

Related U.S. Application Data
(60) Provisional application No. 63/011,224, filed on Apr. 16, 2020.

(51) Int. Cl. A61K 9/00; A61K 47/10; A61K 31/4375; A61K 47/12; A61K 47/18; A61K 47/02; A61K 9/08; A61K 47/14

(52) U.S. Cl. CPC A61K 9/0078; A61K 47/10; A61K 31/4375; A61K 47/12; A61K 47/14; A61K 9/0043; A61K 47/02; A61K 9/08; A61K 47/186

(57) ABSTRACT

The present invention relates to a liquid, propellant-free pharmaceutical formulation and a method for administering the pharmaceutical formulation by nebulizing the pharmaceutical formulation with an inhaler. The propellant-free pharmaceutical formulation comprises: (a) the active substance tiotropium bromide; (b) a solvent; (c) a pH adjusting agent, and optionally other pharmacologically acceptable additives.

### PRIORITY STATEMENT

This application claims the benefit of the filing date of U.S. Provisional Patent Application No. 63/011,224, filed on Apr. 16, 2020, the contents of which are incorporated herein by reference in its entirety.

### BACKGROUND OF THE INVENTION

Tiotropium bromide monohydrate is chemically described as (1α, 2β, 4β, 5α, 7β)-7-[(Hydroxydi-2-thienylacetyl)oxy]-9,9-dimethyl-3-oxa-9-azoniatricyclo[3.3.1.0^{2,4}] nonane bromide monohydrate, and has the following chemical structure:

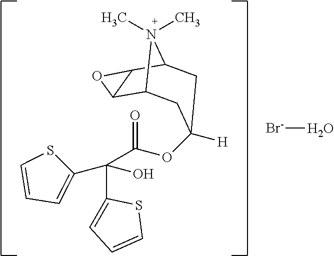

Tiotropium is a long-acting, muscarinic antagonist which is often referred to as an anticholinergic. It has similar affinity for subtypes M1 to M5 of the muscarinic receptor. In the airways, it exhibits a pharmacological effect through inhibition of the M3-receptors on the smooth muscle, leading to bronchodilation. The competitive and reversible nature of antagonism was shown with human and animal origin receptors and isolated organ preparations.

Tiotropium can provide therapeutic benefit in the treatment of asthma or chronic obstructive pulmonary disease, including chronic bronchitis and emphysema.

The present invention relates to a propellant-free inhalable formulation of a pharmaceutically acceptable salt of Tiotropium dissolved in water, in combination with inactive ingredients, preferably administered using a nebulization inhalation device, and the propellant-free inhalable aerosols resulting therefrom.

The pharmaceutical formulations disclosed of the current invention are especially suitable for administration by nebulization inhalation, which provides much better lung deposition (typically up to 55-60%) compared to administration by drying powder inhalation or pMDI.

The pharmaceutical formulations of the present invention are particularly suitable for administering the active substances by nebulization inhalation, especially for treating asthma and chronic obstructive pulmonary disease.

### SUMMARY OF THE INVENTION

The present invention relates to pharmaceutical formulations of Tiotropium and its pharmaceutically acceptable salts or solvates which can be administered by nebulization inhalation. The pharmaceutical formulations according to the invention meets high quality standards.

One aspect of the present invention is to provide an aqueous pharmaceutical formulation containing Tiotropium, which meets the high standards needed in order to achieve optimal nebulization of a solution using the inhalers mentioned hereinbefore. The stability of the formulation is a storage time of some years. In one embodiment, the formulation is stable for at least one year. In one embodiment, the formulation is stable for at least three years.

Another aspect of the invention is to provide propellant-free formulations that are solutions containing Tiotropium which are nebulized under pressure using an inhaler devise, preferably a nebulization inhaler device, wherein the composition is delivered as an aerosol that falls reproducibly within a specified range.

Another aspect of the invention is to provide pharmaceutical formulations that are solutions comprising Tiotropium and other inactive excipients which can be administered by nebulization inhalation using an ultra-sonic based or air pressure based nebulizer/inhaler. The stability of the formulation is a storage time of a few months or years. In one embodiment, the formulation has a storage time of 1-6 months. In one embodiment, the formulation has a storage time of at least one year. In one embodiment, the formulation has a storage time of at least three years.

More specifically, another aspect is to provide a stable pharmaceutical formulation that is an aqueous solution containing Tiotropium and other excipients which can be administered by nebulization inhalation using an ultrasonic jet or mesh nebulizer. The inventive formulation has substantial long term stability. In one embodiment, the formulations have a storage time of at least about 6-24 months at a temperature of from about 15° C. to about 25° C.

### DETAILED DESCRIPTION OF THE INVENTION

It is advantageous to use a liquid formulation without a propellant gas to administer an active substance using a suitable inhaler, in order to achieve a better distribution of the active substance in the lung. Furthermore, it is very important to increase the lung deposition of drugs delivered by inhalation.

Currently, traditional pMDI or DPI (drying powder inhalation) can only delivery about 20-30% of the drug into the lung, resulting in a significant amount of drug being deposited on the month and throat, which can lead to drug entering the stomach and causing unwanted side effects and/or secondary absorption through the oral digestive system.

Therefore, there is a need to improve the inhalation drug delivery by significantly increasing lung deposition.

The pharmaceutical formulations of the invention are a solution that is converted into an aerosol in the nebulizer that is destined for the lungs. The pharmaceutical solution is sprayed with the nebulizer by high pressure.

Nebulization devices useful with the pharmaceutical formulations of the present invention are those in which an amount of less than 8 milliliters of pharmaceutical solution can be nebulized in one puff, preferably less than 2 milliliters, most preferably less than 1 milliliter, so that the inhalable part of aerosol corresponds to the therapeutically effective quantity. In one embodiment, the average particle size of the aerosol formed from one puff is less than about 15 microns. In one embodiment, the average particle size of the aerosol formed from one puff is less than about 10 microns.

The formulations must not contain any ingredients which might interact with the inhaler to affect the pharmaceutical quality of the formulation or of the aerosol produced. In addition, the active substances in the pharmaceutical formulations exhibit very good stability when stored and can be administered directly.

Therefore, one aspect of the present invention is to provide an aqueous pharmaceutical formulation containing Tiotropium, which meets the high standards needed in order to achieve optimum nebulization of the solution using the inhalers mentioned hereinbefore. Preferably the active substances in the pharmaceutical formulation is stable, and has a storage time of some years. In one embodiment, the storage time is at least one year. In one embodiment, the storage time is at least three years.

Another aspect of the current invention is to provide propellant-free formulations that are solutions containing Tiotropium which are nebulized under pressure using an inhaler, preferably a nebulization inhaler, to provide an aerosol, wherein the pharmaceutical formulation delivered by the aerosol falls reproducibly within a specified range.

Another aspect of the invention is to provide an aqueous pharmaceutical formulation that is a solution containing Tiotropium and inactive excipients which can be administered by inhalation.

According to the invention, any pharmaceutically acceptable salt or solvate of Tiotropium may be used for the formulation. When the phrase “Tiotropium salt or solvate” is used herein, it is to be taken as a reference to Tiotropium.

In one embodiment, the active substance is Tiotropium bromide.

In one embodiment, the active substance is Tiotropium bromide monohydrate.

In the formulations according to the invention, Tiotropium is dissolved in a solvent. In one embodiment, the solvent comprises water. In one embodiment, the solvent is water.

In one embodiment according to the invention, a therapeutically effective dose of Tiotropium bromide monohydrate includes from about 3 μg to about 80 μg of Tiotropium bromide monohydrate. In one embodiment, a therapeutically effective dose includes from about 3 μg to about 50 μg of Tiotropium bromide monohydrate. In one embodiment, therapeutically effective dose includes from about 5 μg to about 30 μg of Tiotropium bromide monohydrate.

The concentration of the Tiotropium bromide monohydrate in the finished pharmaceutical formulation depends on the therapeutic effects. The concentration of Tiotropium bromide monohydrate ranges from about 20.7 mcg/100 ml to about 207 mg/100 ml. In one embodiment, the concentration of Tiotropium bromide monohydrate ranges from about 207 mcg/100 ml to about 2.07 mg/100 ml.

In the formulations according to the invention, if desired, the pH can be adjusted by adding a pH adjusting agent to the formulation. In one embodiment, the pH adjusting agent is hydrochloric acid and/or sodium hydroxide.

Other comparable pH adjusting agents include, but are not limited to, citric acid and/or its salts.

The pH is selected to maintain stability of the active ingredients. In one embodiment, the pH ranges from about 1.0 to about 5.0. In one embodiment, the pH ranges from about 2.5 to about 3.5. In one embodiment, the pH ranges from about 2.7 to about 3.1.

If desired, a stabilizer or complexing agent can be included in the formulations. Suitable stabilizers or complexing agents include, but are not limited to, edetic acid (EDTA) or one of the known salts thereof, e.g., disodium edetate or edetate disodium dihydrate. In one embodiment the formulation contains edetic acid and/or a salt thereof.

Other comparable stabilizers or complexing agents can be used in the present invention. Suitable stabilizers or complexing agents include, but are not limited to, citric acid, edetate disodium, and edetate disodium dihydrate.

The phrase “complexing agent,” as used herein, means a molecule which is capable of entering into complex bonds. Preferably, these compounds have the effect of complexing cations. The concentration of the stabilizers or complexing agents ranges from about 1 mg/100 ml to about 500 mg/100 ml. In one embodiment, the concentration of the stabilizers or complexing agents ranges from about 10 mg/100 ml to about 200 mg/100 ml. In one embodiment, the stabilizer or complexing agent is edetate disodium dihydrate at a concentration ranging from about 1 mg/100 ml to about 500 mg/100 ml.

In the formulations according to the invention, if desired, the isosmotic status of the formulation can be adjusted by adding an isosmotic adjusting agent, such as sodium chloride. In one embodiment, the isosmotic adjusting agent is sodium chloride.

In one embodiment, the quantity of sodium chloride is from about 0.8% (w/w) to about 1.0% w/w). In one embodiment, the quantity of sodium chloride is about 0.9% (w/w).

In the formulations according to the invention, the formulation can contain a preservative. In one embodiment, the preservative is selected from the group consisting of benzalkonium chloride, benzoic acid, sodium benzoate, and combinations thereof.

In one embodiment, the Tiotropium bromide is present in solution.

In one embodiment, all the ingredients of the formulation are present in solution.

The term “additive,” as used herein means any pharmacologically acceptable and therapeutically useful substance which is not an active substance, but can be formulated together with the active substances in a pharmacologically suitable solvent, in order to improve the qualities of the formulation. Preferably, these substances have no pharmacological effects or no appreciable pharmacological effects, or at least no undesirable pharmacological effects in the context of the desired therapy.

Suitable additives include, but are not limited to, other stabilizers, complexing agents, antioxidants, surfactants, and/or preservatives which prolong the shelf life of the finished pharmaceutical formulation, vitamins, and/or other additives known in the art.

The pharmaceutical formulation solution is converted by the nebulizer into aerosol destined for the lungs. The pharmaceutical solution is sprayed with the nebulizer by high pressure.

### EXAMPLES

Materials and Reagents:
Tiotropium bromide monohydrate, from Anovent Pharmaceutical Co., Ltd. in Nanchang, China
Sodium chloride, from Merck
Citric acid, from Merck
Sodium hydroxide, from Titan Reagents Co., Ltd. in Shanghai, China
Hydrochloric acid, from Titan Reagents Co., Ltd. in Shanghai, China 50% benzalkonium chloride (referred to as BAC) aqueous solution is commercially available and may be purchased from Spectrum Pharmaceuticals Inc.
Edetate disodium dehydrate is commercially available and may be purchased from purchased from Merck & Co.

### Example 1

The preparation of sample I, sample II, and sample III inhalation solutions is as follows: active and inactive ingredients according to the amounts provided in table 1 were dissolved in 90 ml of purified water and the pH adjusted to the target pH with hydrochloric acid or sodium hydroxide. Purified water was then added to a final volume of 100 ml.

TABLE 1
Ingredient Contents of Sample I, Sample II, and
Sample III of a 100 ml Inhalable Formulation

| | | | |
|---|---|---|---|
| Ingredients | Sample I | Sample II | Sample III |

| | | | | | | |
|---|---|---|---|---|---|---|
| Tiotropium bromide | 20.7 | mcg | 2.07 | mg | 207 | mg |
| monohydrate | | | | | | |
| Sodium chloride | 0.8 | g | 0.9 | g | 1.0 | g |

| | | | |
|---|---|---|---|
| hydrochloric acid or | To pH 1.0 | To pH 3.0 | To pH 5.0 |
| sodium hydroxide | | | |
| Purified water | Added to | Added to | Added to |
| | 100 ml | 100 ml | 100 ml |

### Example 2

The preparation of sample IV inhalation solution is as follows: active and inactive ingredients according to the amounts provided in table 2 were dissolved in 90 ml of purified water and the pH of the solution adjusted to the target pH with hydrochloric acid or sodium hydroxide. Purified water was then added to a final volume of 100 ml.

TABLE 2
Ingredient Contents of Sample IV
of a 100 ml Inhalable Formulation

| Ingredients | Sample IV | |
|---|---|---|
| Tiotropium bromide monohydrate | 0.620 | mg |
| Sodium chloride | 0.9 | g |

| | |
|---|---|
| Hydrochloric acid | To pH 2.9 |
| Purified water | Added to 100 ml |

### Example 3

Sample IV was sprayed using a nebulization inhaler. A Malvern Spraytec (STP5311) was used to measure the particle size of the resulting droplets.

TABLE 3
Particle Size Distribution of Sample
IV by Using a Nebulization Inhaler

| | |
|---|---|
| Sample Number | Droplet size (μm) |

| | | |
|---|---|---|
| Sample IV | D10 | 2.01 |
| | D50 | 5.13 |
| | D90 | 11.07 |

### Example 4

TABLE 4
Osmotic Pressure of Sample IV Inhalable Formulation

| Sample Number | Osmotic pressure |
|---|---|
| Sample IV | 295 mOsm |

### Example 5

Influence of pH on Stability:

The stability of the formulation is highly dependent on pH. Eight samples were prepared according to Table 5. 180 ml water were adjusted to a pH of 2.7, 3.0, 3.3, 3.6, 3.9, 4.2, and 4.5, respectively with HCl, obtain 7 different pH buffers. The pH of sample 8 was left unadjusted. Tiotropium bromide (referred to as TB) in the amounts provided in Table 5 was dissolved in the 180 ml of each buffer. The resulting mixtures were sonicated until completely dissolved. Purified water was then added to a final volume of 200 ml for each sample.

The formula of samples 1-8 is shown in Table 5. Each Sample was stored at 60° C. for 28 days. Experimental data for the stability of each sample is provided in Table 6-7.

TABLE 5
Formulation Design of TB Screening at Different pH Values

| Ingredients | Sample1 | Sample2 | Sample3 | Sample4 | Sample5 | Sample6 | Sample7 | Sample8 |
|---|---|---|---|---|---|---|---|---|
| Tiotropium bromide | 56 mg | 56 mg | 56 mg | 56 mg | 56 mg | 56 mg | 56 mg | 56 mg |
| monohydrate | | | | | | | | |
| HCl | Adjust to | Adjust to | Adjust to | Adjust to | Adjust to | Adjust to | Adjust to | pH not |
| | pH 2.7 | pH 3.0 | pH 3.3 | pH 3.6 | pH 3.9 | pH 4.2 | pH 4.5 | adjusted |
| Purified water | 200 ml | 200 ml | 200 ml | 200 ml | 200 ml | 200 ml | 200 ml | 200 ml |

Impurity A, CAS number: 4746-63-8

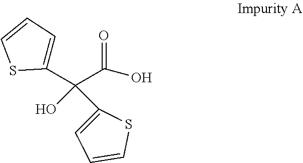 [SMILES: O=C(O)C(O)(c1cccs1)c1cccs1]

Impurity F, CAS number: 704-38-1

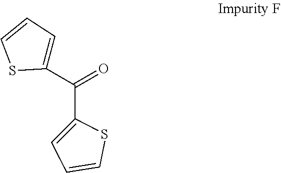 [SMILES: O=C(c1cccs1)c1cccs1]

TABLE 6
Stability at Different pH Values

| | | Impurity percentage | | | |
|---|---|---|---|---|---|
| | | Sample1 | Sample2 | Sample3 | Sample4 |
| | | pH | | | |
| | | 2.7 | 3 | 3.3 | 3.6 |
| 0 day | Impurity A(%) | 0.03 | 0.04 | 0.08 | 0.08 |
| | Impurity F(%) | ND | ND | ND | ND |
| 60° C. | Impurity A(%) | 0.59 | 1.08 | 1.96 | 3.79 |
| 7 days | Impurity F(%) | 0.02 | 0.02 | 0.03 | 0.04 |
| 60° C. | Impurity A(%) | 0.91 | 1.82 | 3.17 | 6.27 |
| 14 days | Impurity F(%) | 0.07 | 0.08 | 0.11 | 0.15 |
| 60° C. | Impurity A(%) | 1.02 | 2.15 | 4.42 | 8.13 |
| 28 days | Impurity F(%) | 0.16 | 0.22 | 0.31 | 0.43 |

ND: not detected

TABLE 7
Stability at Different pH Values

| | | Impurity percentage | | | |
|---|---|---|---|---|---|
| | | Sample5 | Sample 6 | Sample7 | Sample8 |
| | | pH | | | |
| | | | | | pH not |
| | | 3.9 | 4.2 | 4.5 | adjusted |
| 0 day | Impurity A(%) | 0.1 | 0.14 | 0.27 | 1.62 |
| | Impurity F(%) | ND | ND | ND | ND |
| 60° C. | Impurity A(%) | 7.15 | 9.71 | 13.6 | 17.6 |
| 7 days | Impurity F(%) | 0.05 | 0.03 | 0.05 | 0.06 |
| 60° C. | Impurity A(%) | 10.14 | 14.83 | 21.34 | 28.86 |
| 14 days | Impurity F(%) | 0.13 | 0.14 | 0.24 | 0.28 |
| 60° C. | Impurity A(%) | 14.04 | 21.81 | 24.49 | 32.24 |
| 28 days | Impurity F(%) | 0.48 | 0.62 | 0.7 | 0.9 |

ND: not detected

The above results demonstrate that the stability of the Tiotropium bromide solutions are highly dependent on the pH. As can be seen from Table 6 and 7, the Tiotropium bromide solution is stable at pH 2.7 to 3.3.

### Example 6

Influence of EDTA Concentration on Stability:

Four samples were prepared according to Table 8. 50% benzalkonium chloride aqueous solution (referred to as 500% BAC) and edetate disodium dihydrate according to the amounts provided in Table 8 were dissolved in 180 ml of purified water. Samples 9-12 were adjusted to a pH of 2.85 with HCL TB according to the amounts provided in Table 8 was added to each solution and the resulting mixtures sonicated until completely dissolved. Purified water was then added to a final volume of 200 ml for each sample.

The formula for samples 9-12 are provided in Table 8. Each Sample was stored at 60° C. for 28 days. Experimental data for the stability of each sample is provided in Table 9.

TABLE 8
Content of Formulation 9-12

| Ingredients | Sample 9 | Sample 10 | Sample 11 | Sample 12 |
|---|---|---|---|---|
| Tiotropium bromide | 56 mg | 56 mg | 56 mg | 56 mg |
| monohydrate | | | | |
| 50% BAC | 40 mg | 40 mg | 40 mg | 40 mg |
| EDTA | 22 mg | 44 mg | 11 mg | NA |
| HCl | 2.85 | 2.85 | 2.85 | 2.85 |
| Purified water | 200 ml | 200 ml | 200 ml | 200 ml |

TABLE 9
Stability at Different Concentration of EDTA

| | | Impurity percentage | | | |
|---|---|---|---|---|---|
| | | Sample 9 | Sample 10 | Sample 11 | Sample 12 |
| 0 day | Impurity A(%) | ND | ND | ND | ND |
| | Impurity F(%) | ND | ND | ND | ND |
| 60° C. | Impurity A(%) | 0.275 | 0.276 | 0.260 | 0.122 |
| 7 days | Impurity F(%) | 0.008 | 0.007 | 0.008 | 0.022 |
| 60° C. | Impurity A(%) | 0.384 | 0.361 | 0.358 | 0.246 |
| 14 days | Impurity F(%) | 0.018 | 0.018 | 0.022 | 0.043 |
| 60° C. | Impurity A(%) | 0.527 | 0.570 | 0.511 | 0.350 |
| 28 days | Impurity F(%) | 0.059 | 0.043 | 0.044 | 0.092 |

ND: not detected

As can be seen from Table 9, the Tiotropium bromide solution is stable in the EDTA concentration of 0 mg/100 ml to about 22 mg/100 ml.

### Example 7

Aerodynamic Particle Size Distribution:

TABLE 10
Ingredient Contents of Sample 13

| Ingredients | Sample 13 |
|---|---|
| Tiotropium bromide monohydrate | 0.28 g |
| 50% BAC | 0.2 g |
| EDTA | 0.11 g |
| HCl | Adjust to pH 2.85 |
| Purified water | Added to 1000 ml |

Sample 13 solution was prepared in the same way that samples 9-12 were prepared but using the amounts provided in Table 13.

The aerodynamic particle size distribution was determined using an Andersen Scale Impactor (ACI). The inhalation device, named Respimat, was purchased from Boehringer Ingelheim. The Respimat inhaler was held close to the ACI inlet until no aerosol was visible. The flow rate of the ACI was set to 28.3 L/minute and was operated under ambient temperature and a relative humidity (RH) of 90%.

The solution of sample 13 was discharged into the AC. Fractions of the dose were deposited at different stages of the ACI, in accordance with the particle size of the fraction. Each fraction was washed from the stage and analyzed using HPLC.

The results are provided below in Table 11.

TABLE 11
Single Dose Level Distribution and Aerodynamic Particle
Size Distribution of TB Inhalation Formulation Sample
13 Administered by Respimat Inhalation

| | | | Cut-off |
|---|---|---|---|
| | Dosage | Percentage content | diameter |
| Deposited | mcg | at all levels | (μm) |
| Throat | 1.4114 | 41.79% | |
| Stage 0 | 0.1499 | 4.44% | 9.0 |
| Stage 1 | 0.2268 | 6.71% | 5.8 |
| Stage 2 | 0.2294 | 6.79% | 4.7 |
| Stage 3 | 0.3087 | 9.14% | 3.3 |
| Stage 4 | 0.1314 | 3.89% | 2.1 |
| Stage 5 | 0.0840 | 2.49% | 1.1 |
| Stage 6 | 0.2143 | 6.34% | 0.7 |
| Stage 7 | 0.3210 | 9.50% | 0.4 |
| Stage F | 0.3008 | 8.91% | |
| Theoretical | 3.094 | | |
| dose(μg) | | | |
| Actual test | 3.3777 | | |
| dose(μg) | | | |
| Recovery | 109.2% | | |
| rate % | | | |
| Fine Particle | 47.06% | | |
| Fraction (FPF) | | | |

The larger the FPF value, the higher the atomization efficiency.

The above results demonstrate that the formulation of the present invention has a good atomization effect.

### Example 8

Stability Experiment.

TABLE 12
Ingredient Contents of Sample 14-16

| Ingredients | Sample 14 | Sample 15 | Sample 16 |
|---|---|---|---|
| Tiotropium bromide | 56 mg | 56 mg | 56 mg |
| monohydrate | | | |
| 50% BAC | 40 mg | 40 mg | 40 mg |
| EDTA | 22 mg | 22 mg | 22 mg |
| HCl | Adjust to | Adjust to | Adjust to |
| | pH 2.75 | pH 2.85 | pH 2.95 |
| Purified water | Added to | Added to | Added to |
| | 200 ml | 200 ml | 200 ml |

Sample 14-16 were prepared in the same way that samples 9-12 were prepared, but using the amounts provided in Table 12.

Samples 14-16 were stored at 40° C./75% RH for 0, 1, 2, 3, and 6 months. The impurity profile at each time point is provided below in Tables 13-14.

TABLE 13
Content result of Sample 14-16 (Conditions:
40° C. ± 2° C./75% ± 5% RH)

| | percentage (%) | | |
|---|---|---|---|
| Samples | TB | 50% BAC | EDTA |
| Sample 14-0 Month | 99.7 | 103.0 | 100.6 |
| Sample 15-0 Month | 99.5 | 112.6 | 101.0 |
| Sample 16-0 Month | 99.7 | 105.1 | 101.0 |
| Sample 14-1 Month | 99.5 | 103.1 | 99.3 |
| Sample 15-1 Month | 99.4 | 113.4 | 100.6 |
| Sample 16-1 Month | 99.5 | 106.0 | 100.3 |
| Sample 14-2 Months | 98.6 | 104.5 | 99.05 |
| Sample 15-2 Months | 98.3 | 113.8 | 98.89 |
| Sample 16-2 Months | 98.2 | 106.4 | 98.02 |
| Sample 14-3 Months | 100.1 | 103.4 | 99.0 |
| Sample 15-3 Months | 97.6 | 113.0 | 99.6 |
| Sample 16-3 Months | 98.9 | 104.6 | 99.8 |
| Sample 14-6 Months | 99.5 | 100.9 | 102.1 |
| Sample 15-6 Months | 98.7 | 110.1 | 102.5 |
| Sample 16-6 Months | 97.8 | 103.4 | 101.5 |

TABLE 14
The Stability Results of Sample 14-16 (Conditions:
40° C. ± 2° C./75% ± 5% RH)

| | | Impurity percentage | | |
|---|---|---|---|---|
| | | Sample 14 | Sample 15 | Sample 16 |
| 0 month | Impurity A(%) | ND | ND | ND |
| | Impurity F(%) | ND | ND | ND |
| 1 month | Impurity A(%) | 0.130 | 0.141 | 0.192 |
| | Impurity F(%) | 0.001 | 0.001 | 0.001 |
| 2 months | Impurity A(%) | 0.225 | 0.250 | 0.360 |
| | Impurity F(%) | 0.004 | 0.004 | 0.006 |
| 3 months | Impurity A(%) | 0.275 | 0.315 | 0.454 |
| | Impurity F(%) | 0.006 | 0.006 | 0.007 |
| 6 months | Impurity A(%) | 0.378 | 0.449 | 0.674 |
| | Impurity F(%) | 0.015 | 0.025 | 0.026 |

ND: not detected

As shown in Tables 13-14, at pH 2.75-2.95 the TB solutions exhibited good stability. TB solutions ranging from a pH of about 2.75 to about 2.95 were stable for about 6 months at 40° C.±2° C./7500±500 RH.

While various embodiments of the present invention have been described above, it should be understood that they have been presented by way of example only, and not limitation. For example, the present invention is not limited to the physical arrangements or dimensions illustrated or described. Nor is the present invention limited to any particular design or materials of construction. As such, the breadth and scope of the present invention should not be limited to any of the above-described exemplary embodiments, but should be defined only in accordance with the following claims and their equivalents.

What is claimed is:

1. A propellant-free inhalation formulation comprising Tiotropium or a salt or solvate thereof, a pH adjusting agent, and a pharmacologically acceptable additive dissolved in a solvent.

2. The formulation of claim 1, wherein the Tiotropium or a salt thereof is Tiotropium bromide.

3. The formulation of claim 1, wherein the Tiotropium or a salt or solvate thereof is Tiotropium bromide monohydrate.

4. The formulation according to claim 3, wherein Tiotropium bromide monohydrate is present in an amount ranging from about 20.7 mcg/100 ml to about 207 mg/100 ml.

5. The formulation of claim 1, further comprising an isosmotic adjusting agent selected from the group consisting of sodium chloride, glucose, mannitol, glucitol, and combinations thereof.

6. The formulation of claim 1, further comprising an isosmotic adjusting agent in an amount ranging from about 0.8% (w/w) to about 1% (w/w).

7. The formulation of claim 1, wherein the solvent comprises water.

8. The formulation of claim 1, wherein the solvent is water.

9. The formulation of claim 1, further comprising a pH adjusting agent selected from the group consisting of citric acid-citrate, citric acid, hydrochloric acid, and sodium hydroxide.

10. The formulation of claim 1, wherein the formulation has a pH ranging from about 1.0 to about 5.0.

11. The formulation of claim 1, wherein the formulation has a pH ranging from about 2.5 to about 3.5.

12. The formulation of claim 1, wherein the formulation has a pH ranging from about 2.7 to about 3.1.

13. The formulation of claim 1, wherein the pharmacologically acceptable additive is selected from the group consisting of edetic acid, edetate disodium dihydrate, edetate disodium, citric acid, and combinations thereof.

14. The formulation of claim 1, wherein the pharmacologically acceptable additive is selected from the group consisting of benzalkonium chloride, benzoic acid, sodium benzoate, and combinations thereof.

15. The formulation of claim 1, wherein the pharmacologically acceptable additive is present in an amount ranging from about 1 mg/100 ml to about 500 mg/100 ml.

16. The formulation of claim 1, wherein the formulation has an osmotic pressure ranging from about 100 mOsm to about 400 mOsm.

17. A method of treating asthma or COPD in a patient, comprising administering to the patient the pharmaceutical formulation of claim 1 by inhalation.

18. The method of claim 17, wherein the Tiotropium or a salt thereof is Tiotropium bromide monohydrate and the Tiotropium bromide monohydrate is administered at a dose ranging from about 3 μg to about 80 μg.

19. The method of claim 18, wherein the Tiotropium bromide monohydrate is administered at a dose ranging from about 5 μg to about 30 μg.

20. The method of claim 17, wherein the pharmaceutical formulation is administered using a nebulization inhalation device to provide an inhalable aerosol of the pharmaceutical formulation.

21. The method of claim 20, wherein the inhalable aerosol has a D50 that is less than about 10 μm.

22. The method of claim 20, wherein the inhalable aerosol has an average particle size of less than about 15 microns.
